SEQUENCE LISTING (4 sequences)

SEQ ID NO: 1 - DNA, 19 bp, Homo sapiens
agtggaacgaaaactcacg

SEQ ID NO: 2 - DNA, 22 bp, Homo sapiens
taagcattggtaactgtcagac

SEQ ID NO: 3 - DNA, 20 bp, Homo sapiens
actcctacgggaggcagcag

SEQ ID NO: 4 - DNA, 17 bp, Homo sapiens
attaccgcggctgctgg

(19) United States
(12) Patent Application Publication
(10) Pub. No.: US 2006/0068430 A1
(43) Pub. Date: Mar. 30, 2006

(54) Purification of biomolecules from contaminating intact nucleic acids

(75) Inventors: Brian Walter Ward, Webster Groves, MO (US); Brian Ward Buntaine, St. John, MO (US)

Correspondence Address: POLSINELLI SHALTON WELTE SUELTHAUS PC, Suite 1100, 100 S. Fourth Street, St. Louis, MO, 63102, US

(73) Assignee: Sigma-Aldrich Co.

(21) Appl. No.: 11/225,960

(22) Filed: Sep. 14, 2005

Related U.S. Application Data
(60) Provisional application No. 60/611,480, filed on Sep. 20, 2004.

(51) Int. Cl. C12Q 1/68; C12N 1/08

(52) U.S. Cl. 435/6; 435/270

(57) ABSTRACT

The present invention provides methods for the removal, destruction or inactivation of intact nucleic acids contaminating desired biomolecules by the use of small molecule nucleic acid cleavage agents.

### CROSS-REFERENCE TO RELATED APPLICATIONS

This application claims priority from Provisional Application Ser. No. 60/611,480 filed on Sep. 20, 2004, which is hereby incorporated by reference in its entirety.

### FIELD OF THE INVENTION

The present invention relates to methods for the removal, destruction or inactivation of intact nucleic acids contaminating desired biomolecules by the use of small molecule nucleic acid cleavage agents.

### BACKGROUND OF THE INVENTION

The invention relates to a method for the removal, destruction or inactivation of intact nucleic acids contaminating desired biomolecules by the use of small molecule nucleic acid cleavage agents.

A variety of techniques may be employed to facilitate the preparation of intracellular proteins from microorganisms. Typically, the initial steps in these techniques involve lysis or rupture of the bacterial cells to disrupt the bacterial cell wall and allow release of the intracellular proteins into the extracellular milieu. Following this release, the desired proteins are purified from the extracts, typically by a series of chromatographic steps.

Several approaches have proven useful in accomplishing the release of intracellular proteins from bacterial cells. Included among these are the use of chemical lysis, physical methods of disruption, or a combination of chemical and physical approaches (reviewed in Felix, H., Anal. Biochem. 120, 211-234 (1982)).

Physical methods, and many chemical techniques, typically result in the release from the cells not only of the desired intracellular proteins, but also of undesired nucleic acids and membrane lipids (the latter particularly resulting when organic solvents are used). These undesirable cellular components also complicate the subsequent processes for purification of the desired proteins, as they increase the viscosity of the extracts (Sambrook, J., et al., in: Molecular Cloning: A Laboratory Manual, 2nd ed., Cold Spring Harbor, N.Y.: Cold Spring Harbor Laboratory Press, p. 17-38 (1989); Cull, M., and McHenry, C. S., Meth. Enzymol. 182, 147-153 (1990)), and bind with high avidity and affinity to nucleic acid-binding proteins such as DNA polymerases, RNA polymerases and restriction enzymes.

One problem associated with these approaches is that the enzyme preparations are typically contaminated with nucleic acids (e.g., RNA and DNA). This contaminating nucleic acid may come not only from the organisms which are the source of the enzyme, but also from unknown organisms present in the reagents and materials used to purify the enzyme after its release from the cells. Since reverse transcriptase enzymes and DNA polymerase enzymes are routinely used in techniques of amplification and synthesis of nucleic acid molecules (e.g., the Polymerase Chain Reaction (PCR)), the presence of contaminating DNA in the enzyme preparations is a significant problem since it can give rise to spurious amplification or synthesis results. Corless et al. discussed problems with nucleic acid contamination in Taq DNA polymerase in the development of real-time universal 16S rRNA PCR (J. Clin. Microbiol., 38(5), 1747-1752 (2000)).

Additionally, the presence of intact nucleic acids is a problem in the preparation and application of biopharmaceuticals. Nucleic acid contamination of the therapeutic agent would introduce extraneous and possibly deleterious sequences into a patient.

Nucleic acid contamination is most notable for DNA, in that it is many times more stable to environmental conditions than is RNA. In principle there are two ways of preparing a biomolecule that is free of quantities of amplification detectable DNA. One may either segregate the biomolecule of interest and DNA using separation techniques such as chromatographic and/or extraction and related methodologies or one may degrade/modify the DNA so that it is no longer a substrate for amplification detection. Both methods are prevalent in the art. That is, DNA is removed from biomolecules by selective adsorption (e.g., membranes, solid supports, chromatographic media and other matrices), selective precipitation or degradation/modification. The prior art by which DNA is removed/deactivated from biomolecules by degradation/modification is hydrolytic and predominately enzymatic. Enzymatic methods include using random cleaving endonucleases (DNase I, Benzonase, modified recombinant DNase I, etc.), restriction endonucleases, and exonucleases. Corless (J. Clin. Microbiology 38, 1747-52 (2000)) also used irradiation and photocrosslinking to render contaminating DNA non-amplifiable.

Existing methods for the preparation of biomolecules substantially free of contaminating intact nucleic acids suffer from several disadvantages, including the difficulty of removing enzymatic reagents from the biomolecule of interest, the time involved in lengthy procedures or the achievement of ultra-high purification.

Thus, a need exists for preparation of biomolecules that are substantially free of contamination by intact nucleic acids.

Small molecule nucleic acid cleavage agents of various kinds have been utilized to degrade DNA for various purposes, but their use has not been demonstrated for the purification of bioactive biomolecules.

Cationic, water-soluble porphyrins have been used as selective oxidants for the cleavage of nucleic acids (Suslick, K. S. In The Porphyrin Handbook; “Shape Selective Oxidations of Metalloporphyrins,” Kadish, K. M., Smith, K. M., Guilard, R., Eds.; Academic Press: New York, 1999).

Sakata et al. describes the selective removal of DNA from protein solution with copolymer particles derived from N,N-dimethylaminopropylacrylamide (J. Chromatog. A, 1030, 117-122 (2004)).

Huang et al. describes the optimization of DNase I removal of contaminating DNA from RNA for use in quantitative RNA-PCR (Biotechniques, 20(6), 1012-1020 (1996)).

Caroll et al. describes the elimination of bacterial DNA from Taq DNA polymerases by restriction endonuclease digestion (J. Clin. Microbiol., 37(10), 3402-3404 (1999)).

U.S. Pat. No. 5,418,149 describes a method of purifying Taq DNA polymerase from contaminating nucleic acids by overexpression in a dut- and UNG-E. coli strain and subsequent treatment with uracil N-glycosylase.

U.S. Pat. No. 5,858,650 describes methods and reagents utilizing metal chelates to inactivate nucleotide sequences, particularly products of polymerase and ligase chain reactions.

U.S. Pat. No. 6,245,533 describes methods for the production of thermostable enzymes, such as DNA polymerases and restriction endonucleases, by permeabilizing bacterial cells to form spheroplasts and isolating the enzymes under conditions favoring their partitioning from nucleic acids.

U.S. Pat. No. 6,541,204 discloses a method for removing nucleic acid contamination in an amplification reaction which comprises the use of a thermolabile DNAase.

U.S. 2002/0172972 describes a method for rendering contaminating nucleic acids inert by utilizing an activated nucleic acid digestion enzyme, followed by treatment with an inactivating agent.

WO 03/087402 describes a method for the preparation of reagents for amplification of nucleic acids that exhibit no significant contamination by nucleic acids through ultra-violet treatment in the presence of photoactive reagents.

U.S. Pat. No. 6,331,393 describes a method for determining sites of cytosine methylation utilizing bisulfite to effect cytosine deamination.

Chen and Shaw describe the use of bisulfite to convert cytosine to uracil in double stranded DNAs under physiologically relevant conditions (Biochemistry 33, 4121-4129 (1994)).

A need however remains for an improved method for the purification of biomolecules from contaminating nucleic acids. A special need exists for such a method to provide enzymes utilized in gene amplification procedures that are substantially free of intact nucleic acids that is simpler and faster than existing methods. To address the continuing need for the purification of biomolecules uncontaminated with nucleic acids, methods to achieve that end are herein reported.

### SUMMARY OF THE INVENTION

The present invention provides methods for the removal, destruction or inactivation of intact nucleic acids contaminating desired biomolecules by the use of small molecule nucleic acid cleavage agents.

Among its several embodiments, the present invention provides a method for the isolation of a biomolecule originating from natural, genetically engineered or biotechnological biological sources, which is substantially free of intact nucleic acids, comprising the following steps:

a) treatment of a medium comprising the biomolecule to be isolated with a small molecule nucleic acid cleavage agent; and

b) optionally, purification of the resultant medium comprising the biomolecule to remove cleaved DNA and/or the small molecule nucleic acid cleavage agent.

In another embodiment, the present invention further provides a kit that is suitable for use in the isolation of a biomolecule originating from natural, genetically engineered or biotechnological biological sources, which is substantially free from intact nucleic acids, comprising the following steps:

a) treatment of a medium comprising the biomolecule to be isolated with a solution comprising a small molecule nucleic acid cleavage agent; and

b) optionally, purification of the resultant medium comprising the biomolecule to remove damaged DNA and/or the small molecule nucleic acid cleavage agent;

wherein the kit comprises a small molecule nucleic acid cleavage agent, chromatographic matrices for purification of a biomolecule and aqueous buffer solutions.

Further scope of the applicability of the present invention will become apparent from the detailed description provided below. However, it should be understood that the following detailed description and examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

Definitions

The following definitions are provided in order to aid the reader in understanding the detailed description of the present invention.

As used herein, the term “substantially free of contamination from nucleic acids” means an enzyme composition that comprises no nucleic acids, or that comprises nucleic acids below the level of detection, when assayed by standard biochemical assays for nucleic acids. Such assays may include gel electrophoresis (e.g., agarose gel electrophoresis coupled with nucleic acid staining such as ethidium bromide, acridine orange or Hoechst staining), spectrophotometry (e.g., ultraviolet, atomic absorption, NMR or mass spectrometry), chromatography (liquid, gas, HPLC or FPLC), or by functional assays for nucleic acids detection such as amplification. An example of such a functional assay is based on measuring incorporation of labeled nucleotides (e.g., radio labeled, enzyme labels, chemiluminescent labels, etc.) by the enzyme preparation in a “no template” nucleic acid amplification reaction.

The term “intact nucleic acids” means nucleic acids that can function as templates for replication, transcription, or translation.

The term “BAC” (Bacterial Artificial Chromosome) describes a cloning vector based on bacterial mini-F plasmids.

The term “EDTA” refers to ethylenediaminetetraacetic acid.

The term “MTP” refers to metallo tetrapyridylporphyrin.

The term “MnTP” refers to manganese tetrapyridylporphyrin.

The term “FeTP” refers to iron tetrapyridylporphyrin.

The term “MPE” refers to methidiumpropyl EDTA.

The term “FeMPE” refers to iron methidiumpropyl EDTA.

The term “Oxone®” refers to potassium peroxymonosulfate, KHSO₅ [CAS-RN 10058-23-8].

The term “UNG” refers to uracil-DNA N-glycosylase.

The term “amplification” refers to any in vitro means for increasing the number of copies of a target sequence of nucleic acid. Methods include but are not limited to PCR (Polymerase Chain Reaction) and modifications thereto, LAR (Ligase Amplification Reaction) or LCR (Ligase Chain Reaction) and RT-PCR (Reverse Transcriptase-PCR). Methods may result in a linear or exponential increase in the number of copies of the target sequence.

The term “DNase” refers to an enzyme which hydrolyzes a phosphodiester bond in the DNA backbone and is not nucleotide sequence specific.

The term “comprising” means “including the following elements but not excluding others.”

Methods

Among its several embodiments, the present invention provides a method for the isolation of a biomolecule originating from natural, genetically engineered or biotechnological biological sources, which is substantially free of intact nucleic acids, comprising the following steps:

a) treatment of a medium comprising the biomolecule to be isolated with a small molecule nucleic acid cleavage agent; and

b) optionally, purification of the resultant medium comprising the biomolecule to remove cleaved DNA and/or the small molecule nucleic acid cleavage agent.

Any standard chromatographic technique can be used to separate the small molecule nucleic acid cleavage agent from the biomolecule of interest by taking advantage of the characteristics of both. For example, Taq DNA polymerase may be purified from manganese tetrapyridylporphyrin (MnTP) by the use of size exclusion chromatography such as Sephadex G-50 resin or by the use of ion exchange resins, such as Amberlite CG50 (weakly acidic cation exchange resin), Dowex 50Wx4-100 (strongly acidic cation exchange resin) or Macro Prep High S Support (strong cation exchange resin).

In another embodiment, the source of the biomolecule to be isolated is a bacterial cell or a recombinant bacterial cell.

In yet another embodiment, the biomolecule to be isolated is a protein.

The method has particular advantages for the expression and purification of proteins used in amplification systems; such proteins include DNA polymerase (e.g., Taq DNA polymerase), DNA ligase, RNA ligase, reverse transcriptase, RNA replicase, RNA polymerase, RNAse or RNAsin. These proteins can be purified prior to their use in PCR or other amplification systems with the present invention. Other processes in the literature for inactivation of nucleic acid templates focus on carryover products from previous PCR or LCR amplifications of the same or similar targets.

Since thermophilic enzymes such as DNA polymerases and restriction enzymes are routinely used in automated techniques of DNA amplification and sequencing, e.g., the Polymerase Chain Reaction (PCR), the presence of contaminating DNA in the enzyme preparations is a significant problem since it can give rise to spurious amplification or sequencing results. Thus, a need exists for preparations of thermostable enzymes that are substantially free of contamination by nucleic acids.

DNA polymerases that may be purified according to the present invention, include, for example, prokarotic DNA polymerase (I, II, or III), T4 DNA polymerase, T7 DNA polymerase, Klenow fragment, Vent DNA polymerase (Vent is a trademark of New England BioLabs, Beverly, Mass.), Thermus thermophilus DNA polymerase, Thermococcus kodakaraenis DNA polymerase, Pfu DNA polymerase, Taq DNA polymerase, and the like, derived from any source such as cells, bacteria (for example, E. coli), plants, animals, virus, thermophilic bacteria, and so forth.

The invention is directed preferably to methods wherein the enzyme being purified is a thermostable DNA polymerase, preferably Taq DNA polymerase, Tne DNA polymerase, Tma DNA polymerase, or a derivative or fragment thereof.

Even more preferably, the DNA polymerase to be isolated by the methods of the present invention is Taq DNA polymerase.

It has been reported by at least two different groups that commercially available preparations of Taq DNA polymerase are contaminated with bacterial DNA (Rand, K. H., and Houck, H., Mol. Cell Probes 4(6), 445-450 (1990); Hughes, M. S., et al., J. Clin. Microbial. 32(8), 2007-2008 (1994)), despite the use of gentle lysis procedures to liberate the enzyme from the cells. Furthermore, this contaminating DNA may come not only from the organisms which are the source of the enzyme (Thermus aquaticus for natural Taq polymerase; E. coli for recombinant enzyme), but also from unknown organisms present in the reagents and materials used to purify the enzyme after its release from the cells (Rand, K. H., and Houck, H., Mol. Cell Probes 4(6), 445450 (1990); Hughes, M. S., et al., J. Clin. Microbio. 132(8), 2007-2008 (1994)). Since thermophilic enzymes such as DNA polymerases and restriction enzymes are routinely used in automated techniques of DNA amplification and sequencing, e.g., the Polymerase Chain Reaction (PCR), the presence of contaminating DNA in the enzyme preparations is a significant problem since it can give rise to spurious amplification or sequencing results. Thus, a need exists for preparations of thermostable enzymes that are substantially free of contamination by nucleic acids.

Enzymes purified in accordance with the present invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, and mutants, fragments, variants or derivatives thereof (see WO 98/147912, U.S. Pat. Nos. 5,668,005, and 5,017,492). As will be understood by one of ordinary skill in the art, modified reverse transcriptases may be obtained by recombinant or genetic engineering techniques that are routine and well known in the art. Mutant reverse transcriptases can, for example, be obtained by mutating the gene or genes encoding the reverse transcriptase of interest by site-directed or random mutagenesis. Such mutations may include point mutations, deletion mutations and insertional mutations. Preferably, one or more point mutations (e.g., substitution of one or more amino acids with one or more different amino acids) are used to construct mutant reverse transcriptases of the invention. Fragments of reverse transcriptases may be obtained by deletion mutation by recombinant techniques that are routine and well-known in the art, or by enzymatic digestion of the reverse transcriptase(s) of interest using any of a number of well-known proteolytic enzymes.

Preferred enzymes which may be prepared according to the invention include those that are reduced or substantially reduced in RNase H activity. Such enzymes that are reduced or substantially reduced in RNase H activity may be obtained by mutating the RNase H domain within the reverse transcriptase of interest, preferably by one or more point mutations, one or more deletion mutations, and/or one or more insertion mutations as described above. By an enzyme “substantially reduced in RNase H activity” is meant that the enzyme has less than about 30%, less than about 25%, less than about 20%, more preferably less than about 15%, less than about 10%, less than about 7.5%, or less than about 5%, and most preferably less than about 5% or less than about 2% of the RNase H activity of the corresponding wild type or RNase H+ enzyme such as wild type Moloney Murine Leukemia Virus (M-MLV), Avian Myeloblastosis Virus (AMV) or Rous Sarcoma Virus (RSV) reverse transcriptases. The RNase H activity of any enzyme may be determined by a variety of assays, such as those described, for example, in U.S. Pat. No. 5,244,797, in Kotewicz, M. L., et al., Nucl. Acids Res. 16, 265 (1988), in Gerard, G. F., et al., FOCUS 14(5), 91 (1992), in WO 98/47912, and in U.S. Pat. No. 5,668,005, the disclosures of all of which are fully incorporated herein by reference.

Particularly preferred enzymes for use in the invention include, but are not limited to M-MLV H-reverse transcriptase, RSV H-reverse transcriptase, AMV H-reverse transcriptase, RAV H-reverse transcriptase, MAV reverse transcriptase and HIV H-reverse transcriptase (see WO 98/147912). It will be understood by one of ordinary skill, however, that any enzyme capable of producing a DNA molecule from a ribonucleic acid molecule (i.e., having reverse transcriptase activity) that is reduced or not reduced in RNase H activity may be equivalently prepared in accordance with the invention.

Amplification methods in which the present enzymes may be used include PCR (U.S. Pat. Nos. 4,683,195 and 4,683,202), Strand Displacement Amplification (SDA, U.S. Pat. No. 5,455,166; EP 0 684 315), and Nucleic Acid Sequence-Based Amplification (NASBA, U.S. Pat. No. 5,409,818; EP 0 329 822). Nucleic acid sequencing techniques which may employ the present enzymes include dideoxy sequencing methods such as those disclosed in U.S. Pat. Nos. 4,962,022 and 5,498,523, as well as more complex PCR-based nucleic acid fingerprinting techniques such as Random Amplified Polymorphic DNA (RAPD)) analysis (Williams, J. G. K., et al., Nucl. Acids Res. 18(22), 6531-6535, 1990), Arbitrarily Primed PCR (AP-PCR; Welsh, J., and McClelland, M., Nucl. Acids Res. 18(24), 7213-7218, 1990), DNA Amplification Fingerprinting (DAF; Caetano-Anolles et al., BiofTechnology 9, 553-557, 1991) microsatellite PCR or Directed Amplification of Minisatellite-region DNA (DAMD; Heath, D. D., et al., Nucl. Acids Res. 21(24), 5782-5785, 1993), and Amplification Fragment Length Polymorphism (AFLP) analysis (EP 0 534 858; Vos, P., et al., Nucl. Acids Res. 23(21), 4407-4414, 1995; Lin, J. J., and Kuo, J., FOCUS 17(2), 66-70, 1995). In particular, the enzymes and kits of the present invention will be useful in the fields of medical therapeutics and diagnostics, forensics, and agricultural and other biological sciences, in any procedure utilizing reverse transcriptase, DNA polymerase or other amplification enzymes.

The contaminating nucleic acids which the present invention removes from the biomolecule of interest may be selected from the group consisting of single-stranded DNA, double-stranded DNA, DNA fragments, oligonucleotides, amplified DNA, BACs and plasmid DNA.

In the method of the present invention, a medium comprising the biomolecule to be isolated is treated with a small molecule cleavage agent. A small molecule cleavage agent is a molecule with a molecular weight less than about 5 kDa. The small molecule nucleic acid cleavage agent is selected from the group consisting of base cleavage agents and backbone cleavage agents.

A base cleavage agent cleaves the nucleic acid bases from the DNA strand and results in non-amplifiable products. Examples of base cleavage agents may be selected from the group consisting of formic acid, dimethyl sulfate, hydrazine, bisulfite+UNG, hydroxylamine, potassium permanganate and osmium tetroxide. In general, the base cleavage agents chemically modify the nucleic acid bases, which are subsequently or concurrently cleaved under specified conditions yielding abasic sites. For example, treatment of DNA with formic acid results in depurination. Methylation of DNA with dimethyl sulfate leads to base cleavage at deoxyguanine. Treatment of DNA with hydrazine opens and cleaves pyrimidine rings. Incubation of DNA with osmium tetroxide or hydroxylamine results in pyrimidine cleavage. Cleavage of the DNA base thymidine can be effected with potassium permanganate. Base treatment, typically piperidine, causes hydrolysis of the phosphodiester backbone at the resultant abasic sites.

Bisulfite deaminates cystosine residues, effectively converting dCG to dUG base pairs, that is converting cystosine to uracil. Although methods utilizing bisulfite for determination of cytosine methylation may be harsh for many biopreparations, the same transformation may be accomplished under physiologically relevant conditions with longer reaction times, thus converting dC to dU in double stranded DNAs. Reaction of the resultant dU containing DNAs with uracil-DNA N-glycosylase (UNG) results in the conversion of the dU sites to abasic sites. DNAs containing substantial quantities of abasic sites are not suitable templates for primer extension reactions and do not function in amplification reactions. Thus, the use of bisulfite followed by UNG treatment provides a method for producing a bioproduct free from amplifiable quantities of contaminating DNA.

A backbone cleavage agent attacks the deoxyribose moiety on the nucleic acid, resulting in cleavage of the nucleic acid chain. A preferred example of a backbone cleavage agent comprises a DNA-binding metal chelating agent. DNA-binding metal chelating agents that are used as footprinting agents are useful as backbone cleavage agents in the present invention.

Footprinting is a method for visualizing protein-DNA binding. In footprinting methods, the DNA of interest is first radioactively end-labeled. The labeled DNA and the protein that binds to it are equilibrated together. Then the DNA-binding metal chelating agent is added. Under the appropriate conditions, the DNA backbone is cleaved, generally by a hydroxyl radical. The presence of the DNA-binding protein prevents the cleavage agent from attacking the DNA backbone that is shielded by bound protein. In the absence of protein, the DNA would be cleaved at every base. The reaction products of the cleavage reaction with and without added protein are visualized by electrophoresis, allowing a direct determination of the particular bases where the protein binds.

Preferably, the DNA-binding metal chelating agent is selected from the group consisting of porphyrins, planar bis-N-donor heterocyclic bases, metal chelator tethered intercalators and natural product small molecules.

In one embodiment, the DNA-binding metal chelating agent is a porphyrin. Examples of porphyrins useful as DNA-binding metal chelating agents in the present invention, include, but are not limited to meso-tetra (6-methyl-N-methyl-2-pyridyl) porphyrin, meso-α,β,γ-tritolyl-δ-(N-methyl-4-pyridiniumyl)porphyrin(1+), meso-α,β-ditolyl-γ, δ-(N-methyl-4-pyridiniumyl)porphyrin(cis-2+), meso-α,γ-ditolyl-β, δ-di(N-methyl-4-pyridiniumyl)porphyrin(trans-2+), meso-α-tolyl-β,γ,δ-tri(N-methyl-4-pyridiniumyl)porphyrin(3+) and tetra (N-methyl-4-pyridyl)porphyrin. The preparation of meso-tetra(6-methyl-N-methyl-2-pyridyl)porphyrin and the toluene-pyridinium series of compounds is described by Bromley, S., et al., in Nucleic Acids Res., 14(22), 9133 (1986).

Preferably, the porphyrin is tetra(N-methyl-4-pyridyl) porphyrin (Ward, B., et al., Biochemistry 25, 6879 (1986)).

In another embodiment, the DNA-binding metal chelating agent is a bis-N-donor heterocyclic base. Examples of planar bis-N-donor heterocyclic bases useful in the present invention, include, but are not limited to 1,10-phenanthroline, dipyridoquinoxaline and dipyridophenazine. The bleomycin mimic, FTP1, whose preparation is described by Searcey, M., et al. in J. Chem. Soc., Perkin Trans. 2, 523 (1997), is another example of a bis-N-donor heterocyclic base.

In yet another embodiment, the DNA-binding metal chelating agent is a metal chelator tethered intercalator. A metal chelator tethered intercalator is a small molecule comprising a metal chelator moiety and a DNA intercalator moiety tethered together by one or more molecular chains. Examples of metal chelator tethered intercalators useful in the present invention, include, but are not limited to acridine porphyrins, acodazole porphyrins and methidiumpropyl EDTA. Methidiumpropyl EDTA may be prepared as described by Hertzberg, R. P. and Dervan, P. B. in Biochemistry 23, 3934 (1984) and Van Dyke, M. W. and Dervan, P. B. in Biochemistry 22, 2373 (1983).

Examples of acridine porphyrins and acodazole porphyrins useful in the methods of the present invention as metal chelator tethered intercalators are described by Lown, J. W., et al. in Biochemistry, 25, 5111 (1986) and shown below as compounds (1)-(6).

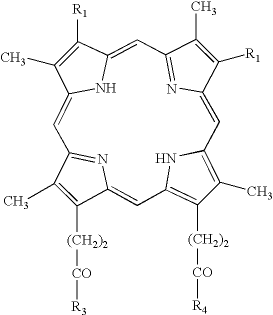

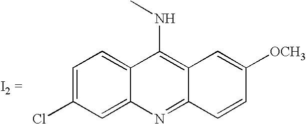

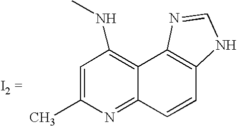

| | | |
|---|---|---|
| (1) R₁ = R₂ = H. | R₃ = OCH₃. | R₄ = —NH(CH₂)₂—I₁ |
| (2) R₁ = R₂ = H. | R₃ = OCH₃. | R₄ = —NH(CH₂)₃—I₁ |
| (3) R₁ = R₂ = H. | R₃ = OCH₃. | R₄ = —NH(CH₂)₃NH(CH₂)₃—I₁ |
| (4) R₁ = R₂ = H. | R₃ = R₄ = —NH(CH₂)₄—I₁ | |
| (5) R₁ = R₂ = H. | R₃ = R₄ = —NH(CH₂)₄—I₂ | |
| (6) R₁ = R₂ = —CH═ CH₂. | R₃ = R₄ = —NH(CH₂)₄—I₂ | |

Other examples of metal chelator tethered intercalators that are of use in the present invention are Mn—P-11-E11 (Pratviel; G., et al., Nucl. Acids Res. 19(22), 6283-6288 (1991)), intercalator tethered deuteroporphyrins (Lown, J. W., et al., Biochemistry 25, 5115-5117 (1986)), intercalator tethered protoporphyrins (Hashimoto, Y., et al., Biochemistry 25, 5103-5110 (1986)), intercalator tethered tetrapyridiniumporphyrins (Ding, L., et al., Biochemistry 29, 7868-7875 (1990)), tentacle porphyrins (Marzilli, L. G., et al., J. Am. Chem. Soc. 114, 7575-7577 (1992)) and copper-bipyridyl porphyrins (Laine, M., et al., J. Biol. Inorg. Chem. 9(5), 550-562 (2004)).

A preferred embodiment of a metal chelator tethered intercalator is methidiumpropyl EDTA.

In still another embodiment, the DNA-binding metal chelating agent is a natural product small molecule. Examples of natural product small molecules useful in the present invention, include, but are not limited to bleomycin, adriamycin, leinamycin, kanamycin, phleomycin and neamine.

In one embodiment of the present invention, the DNA-binding metal chelating agent is complexed to a transition metal. More preferably, the DNA-binding metal chelating agent is complexed to a transition metal selected from the group consisting of iron, copper, manganese, nickel, zinc, ruthenium, rhodium and cobalt.

The planar bis-N-donor heterocyclic bases are preferably complexed to copper (II). The preparation of bis-dipyridoquinoxaline copper (II) and dipyridophenazine copper (II) are described by Chakravarty, A., et al., Proc. Indian Acad. Sci. (Chem. Sci.), 114(4), 391 (2002). Especially preferred in the present invention is copper (II) bis-(1,10-phenanthroline).

In another especially preferred embodiment, the DNA-binding metal chelating agent, tetra(N-methyl-4-pyridyl) porphyrin, is complexed to manganese (III).

In another especially preferred embodiment, the DNA binding metal chelating agent, methidiumpropyl EDTA, is complexed to iron (III).

Examples of preferred DNA-binding metal chelating agents useful in the present invention, include, but are not limited to the compounds listed in Table 1.

TABLE 1
DNA-binding metal chelating agents.

| | CAS | |
|---|---|---|
| | Registry | Preferred |
| Name | Number | Metals |
| Methidiumpropyl-EDTA | 80082-09-3 | Fe (III) |
| meso-Tetra(N-methyl-4- | 36951-72-1 | Mn (III), |
| pyridyl) porphyrin tetratosylate | | Fe (III), |
| salt | | Co (III) |
| 1,10-Phenanthroline | 66-71-7 | Cu (II) |

In another embodiment of the present invention, the small molecule nucleic acid cleavage agent comprises an oxidant. The oxidant is selected, for example, from the group consisting of Oxone®, hydrogen peroxide, molecular oxygen, t-butyl hydroperoxide, peracetic acid, magnesium monoperoxyphthalate, iodosobenzoic acid and persulfate salts. Examples of persulfate salts useful as oxidants in the present invention are, for example, ammonium persulfate, potassium persulfate and sodium persulfate.

In a preferred embodiment, the oxidant is Oxone®. In another preferred embodiment, the oxidant is molecular oxygen. When the oxidant is molecular oxygen in the present invention, a reductant is required to be present. The reductant is selected, for example, from the group consisting of ascorbate salts, 3-mercaptopropionic acid, p-mercaptoethanol and dithiothreitol. When oxidants other than molecular oxygen are used in the present invention, no reductant is required.

In the method of the present invention, the treatment with a backbone cleavage agent preferably comprises the following sequential steps:
a) treatment with a DNA-binding metal chelating agent; and
b) treatment with an oxidant, provided that the oxidant is not molecular oxygen.

For the purification of Taq DNA polymerase, the enzyme must be treated first with the DNA-binding metal chelating agent, followed by treatment with an oxidant, provided that the oxidant is not molecular oxygen. When the oxidant is molecular oxygen, the enzyme may be treated with the DNA-binding metal chelating agent in the presence of molecular oxygen. If the order of the sequential treatment steps is reversed or the treatment steps are simultaneous, in cases where the oxidant is not molecular oxygen, the Taq DNA polymerase is no longer functional in PCR.

In another embodiment, the present invention further provides a kit that is suitable for use in the isolation of a biomolecule originating from natural, genetically engineered or biotechnological biological sources, which is substantially free from intact nucleic acids, comprising the following steps:

a) treatment of a medium comprising the biomolecule to be isolated with a solution comprising a small molecule nucleic acid cleavage agent; and

b) optionally, purification of the resultant medium comprising the biomolecule to remove damaged DNA and/or the small molecule nucleic acid cleavage agent;

wherein the kit comprises a small molecule nucleic acid cleavage agent, chromatographic matrices for purification of a biomolecule and aqueous buffer solutions.

In an embodiment of the present invention, the small molecule nucleic acid cleavage agent contained in the kit comprises a DNA-binding metal chelating agent and an oxidant.

In a preferred embodiment, the DNA-binding metal chelating agent contained in the kit is tetra(N-methyl-4-pyridyl) porphyrin. The tetra (N-methyl-4-pyridyl) porphyrin contained in the kit is preferably complexed to manganese (III). The preferred oxidant contained in the kit is Oxone®.

The compounds useful in the present invention can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

Also included in the methods, combinations and compositions of the present invention are the isomeric forms and tautomers of the described compounds and salts thereof.

The above individual references are each herein individually incorporated by reference.

The present invention is described by reference to the following Examples, which are offered by way of illustration and are not intended to limit the invention in any manner. Standard techniques well known in the art or the techniques specifically described below were utilized.

### EXAMPLES

The following oligonucleotides primers, listed in Table 2, were used in the methods of the present invention.

TABLE NO. 2
Oligonucleotide primers used.

| Oligonucleotide | | |
|---|---|---|
| Primers | | Product |
| (20 μM) | Sequence 5′ to 3′ | Number |
| Expression vector | AGTGGAACGAAAACTCACG | vector F |
| plasmid forward | | |
| Expression vector | TAAGCATTGGTAACTGTCAGAC | vector R |
| plasmid reverse | | |
| Bacterial 16S rRNA | ACTCCTACGGGAGGCAGCAG | Bac16S F |
| forward | | |
| Bacterial 16S rRNA | ATTACCGCGGCTGCTGG | Bac16S R |
| reverse | | |

### PCR Methods

The following PCR methods were used in the methods of the present invention.

PCR Method 1

PCR was performed targeting the Taq expression vector forward and reverse primers. Each 50 μl reaction contained 5 μl of 10× PCR buffer, 1 μl of 10 mM dNTP mix, 1 μl of each forward and reverse pUC19 primers (10 μM each), 1 μl of polymerase (5 units/μl), and 41 μl of water. Cycling conditions were as shown in Table 3. Subsequent 4% Agarose gel electrophoresis was performed to analyze the results.

TABLE 3
Cycling conditions for PCR Methods 1 and 2.

| Temperature (° C.) | Time | Cycles |
|---|---|---|
| 94 | 2 minutes | 1 |
| 94 | 30 seconds | 40 |
| 60 | 30 seconds | |
| 72 | 1 minute | |
| 4 | Hold | hold |

PCR Method 2

PCR was performed targeting the conserved 16S rRNA region of baceria (Bac16S rRNA forward and reverse primers). Each 50 μl reaction contained 5 μl of 10× PCR buffer, 1 μl of 10 mM dNTP mix, 1 μl of each forward and reverse 16S rRNA primers (10 μM each), 1 μl of polymerase (5 units/μl), and 41 μl of water. Cycling conditions were as shown in Table 3. Subsequent 4% Agarose gel electrophoresis was performed to analyze the results.

### Example 1

### Removal/Inactivation of Contaminating DNA from Taq DNA Polymerase with Manganese Tetrapyridylporphyrin and Oxone®

Four aliquots of Taq DNA Polymerase (5 units/μl; Sigma product D1806) were treated with manganese tetrapyridylporphyrin (MnTP) and Oxone® at the final concentrations listed in Table 4 below. MnTP was added first, the solution was gently mixed and allowed to incubate at room temperature for ˜4 minutes. Oxone® was added last, the solution was gently mixed and allowed to incubate at room temperature for 1 hour.

TABLE 4
Concentrations of reagents for treatment of Taq DNA polymerase

| | 1 | 2 | 3 | 4 |
|---|---|---|---|---|
| MnTP (μM) | 0 | 100 | 500 | 1000 |
| Oxone ® (μM) | 0 | 1000 | 1000 | 1000 |

Each sample was then subjected to Sephadex G-50 centrifugal chromatography (Sigma product S5059) and analyzed via PCR Method 1 above. Two reactions were performed with each sample to be tested; one containing no exogenous DNA (no-template control to test contaminant levels) and one containing exogenously added plasmid DNA (positive control to demonstrate the ability to perform PCR).

The following results were obtained. While the no-template reaction from the untreated sample (0 μM MnTP; 0 μM Oxone®) generated PCR product (indicative of contaminant plasmid DNA), none of the three no-template reactions from the three treated samples generated any PCR product (indicative of the destruction/inactivation of all contaminant DNA). All four positive controls generated proper PCR product.

### Example 2

### Removal/Inactivation of Contaminating DNA from Taq DNA Polymerase with Iron Tetrapyridylporphyrin and Oxone®

The methods of Example 1 were repeated exactly, with the substitution of iron tetrapyridylporphyrin for manganese tetrapyridylporphyrin. The results for Example 2 were identical to those of Example 1.

### Example 3

### Removal/Inactivation of Contaminating DNA from Taq DNA Polymerase with Iron Methidiumpropyl EDTA and Oxone®

The methods of Example 1 were repeated exactly, with the substitution of iron methidiumpropyl EDTA for manganese tetrapyridylporphyrin. The results from Example 3 were identical to Example 1 except that PCR Method 2 was also used to analyze treated samples. The no template controls showed no amplification products (indicative of successful removal/inactivation of contaminant DNA), while the positive controls (exogenously-added template) successfully generated the expected product.

### Example 4

### Removal/Inactivation of Contaminating DNA from Taq DNA Polymerase with Iron Methidiumpropyl EDTA (FeMPE) and Ascorbate

The methods of Example 1 were repeated exactly, with the substitution of iron methidiumpropyl EDTA for manganese tetrapyridylporphyrin, and the substitution of ascorbate for Oxone®.

The following results were obtained. The no-template reactions from both the untreated sample (0 μM FeMPE; 0 μM ascorbate) and the 500 μM FeMPE/1 mM ascorbate sample generated PCR products (indicative of contaminant plasmid DNA). Neither of the no-template reactions from the 100 μM FeMPE/1 mM ascorbate nor the 1 mM FeMPE/1 mM ascorbate treated samples generated any PCR product (indicative of the destruction/inactivation of all contaminant DNA). All four positive controls generated proper PCR product. This indicates that the treatment worked, but exogenous contamination of the 500 μM FeMPE/1 mM ascorbate treated sample occurred post-treatment.

### Example 5

### Removal/Inactivation of Contaminating DNA from Taq DNA Polymerase with Iron Tetrapyridylporphyrin (FeTP) and Dithiothreitol (DTT)

To test alternate activation mechanisms for the oxidation/reduction reaction responsible for the destruction/inactivation of contaminant DNA, we tested the use of DTT and molecular oxygen as a method. An aliquot of Taq was treated with 500 μM FeTP and 500 μM DTT for 30 minutes at room temperature. Small molecules were removed exactly as in Example 1 above (Sephadex G50 centrifugal chromatography). PCR Method 1 was used to analyze the samples.

The results are as follows. No-template reactions using untreated Taq generated PCR products (indicative of DNA contaminants). No-template reactions using treated (500 μM FeTP and 500 μM DTT) samples generated no PCR products; positive controls (exogenously-added template) produced PCR product (indicative of destruction/inactivation of contaminant DNA).

### Example 6

### Removal/Inactivation of Contaminating DNA from Taq DNA Polymerase with Manganese Tetrapyridylporphyrin and Iodosobenzoic Acid

The methods of Example 5 were repeated exactly, with the substitution of manganese tetrapyridylporphyrin for iron tetrapyridylporphyrin, and iodosobenzoic acid for DTT.

The results for Example 6 were identical to those from Example 5.

### Example 7

### Removal/Inactivation of Contaminating DNA from Taq DNA Polymerase with Varying the Order of Addition of Reaction Components

To determine the proper order of addition of the reaction components (MnTP versus Oxone®), Taq samples were treated either first with 500 μM Oxone® followed by 500 μM MnTP, or first with 500 μM MnTP followed by 500 μM Oxone®. Small-molecules were removed from each sample exactly as in Example 1 above. Each sample was then analyzed by PCR and subsequent agarose gel electrophoresis for DNA contamination exactly as in Example 1 above. The primers used targeted the Taq expression vector. Two reactions were performed with each sample to be tested; one containing no exogenous DNA (no template control to test contaminant levels) and one containing exogenously added plasmid DNA (positive control to demonstrate the ability to perform PCR). Cycling conditions were as in Example 1 above.

The results from the samples treated with MnTP first were exactly as in Example 1 above. However, none of the reactions from the samples treated by adding Oxone® first performed PCR (demonstrating that the addition of Oxone® prior to MnTP adversely affected the ability of Taq polymerase to perform PCR).

### Example 8

### Removal/Inactivation of Contaminating DNA from Taq DNA Polymerase with Copper bis-(1,10-phenanthroline) Sulfate and Oxone®

Four aliquots of Taq DNA Polymerase (5 units/μl; Sigma product D1806) were treated with copper bis-(1,10-phenanthroline) sulfate and Oxone® at the final concentrations listed in Table 5 below. Copper bis-(1,10-phenanthroline) sulfate was added first, the solution was gently mixed and allowed to incubate at room temperature for ˜4 minutes. Oxone® was added last, the solution was gently mixed and allowed to incubate at room temperature for 1 hour.

TABLE 5
Concentrations of reagents for treatment of Taq DNA polymerase with
copper bis-(1,10-phenanthroline) sulfate.

| | 1 | 2 | 3 | 4 |
|---|---|---|---|---|
| Copper bis-(1,10- | 0 | 100 | 500 | 1000 |
| phenanthroline) | | | | |
| sulfate (μM) | | | | |
| Oxone ® (μM) | 0 | 100 | 500 | 1000 |

Each sample was then subjected to Sephadex G-50 centrifugal chromatography (Sigma product S5059) and analyzed via PCR Method 1 above. Two reactions were performed with each sample to be tested; one containing no exogenous DNA (no-template control to test contaminant levels) and one containing exogenously added plasmid DNA (positive control to demonstrate the ability to perform PCR).

The following results were obtained. While the no-template reaction from the untreated sample (0 μM copper bis-(1,10-phenanthroline) sulfate; 0 μM Oxone®) generated PCR product (indicative of contaminant plasmid DNA), none of the three no-template reactions from the three treated samples generated any PCR product (indicative of the destruction/inactivation of all contaminant DNA). Three of the four positive controls generated proper PCR product. The 1 mM treated samples failed to perform PCR, suggesting that treatment at that level of copper bis-(1, 10-phenanthroline) sulfate and Oxone® adversely affects Taq performance.

The contents of each of the references cited herein, including the contents of the references cited within these primary references, are herein incorporated by reference in their entirety.

While the invention has been described and illustrated with reference to certain particular embodiments thereof, those skilled in the art will appreciate that various changes, modifications and substitutions can be made therein without departing from the spirit and scope of the invention.

It is intended, therefore, that the invention be defined by the scope of the claims which follow and that such claims be interpreted as broadly as is reasonable.

What is claimed is:

1. A method for the isolation of a biomolecule originating from natural, genetically engineered or biotechnological biological sources, which is substantially free of intact nucleic acids, comprising the following steps:
a. treatment of a medium comprising the biomolecule to be isolated with a small molecule nucleic acid cleavage agent; and
b. purification of the resultant medium comprising the biomolecule to remove cleaved DNA and/or the small molecule nucleic acid cleavage agent.

2. The method of claim 1, wherein the source of the biomolecule to be isolated is a bacterial cell or a recombinant bacterial cell.

3. The method of claim 1, wherein the biomolecule to be isolated is a protein.

4. The method of claim 3, wherein the protein to be isolated is selected from the group consisting of DNA polymerases, RNA polymerases and reverse transcriptases.

5. The method of claim 4, wherein the enzyme to be isolated is a DNA polymerase.

6. The method of claim 5, wherein the DNA polymerase to be isolated is Taq DNA polymerase.

7. The method of claim 1, wherein the contaminating nucleic acids are selected from the group consisting of single-stranded DNA, double-stranded DNA, DNA fragments, oligonucleotides, amplified DNA, BACs, and plasmid DNA.

8. The method of claim 1, wherein the small molecule nucleic acid cleavage agent is selected from the group consisting of base cleavage agents and backbone cleavage agents.

9. The method of claim 8, wherein the small molecule nucleic acid cleavage agent is a base cleavage agent.

10. The method of claim 9, wherein the base cleavage agent is selected from the group consisting of formic acid, dimethyl sulfate, hydrazine, bisulfite+UNG, hydroxylamine, potassium permanganate and osmium tetroxide.

11. The method of claim 8, wherein the small molecule nucleic acid cleavage agent is a backbone cleavage agent.

12. The method of claim 11, wherein the backbone cleavage agent is a DNA-binding metal chelating agent.

13. The method of claim 12, wherein the DNA-binding metal chelating agent is selected from the group consisting of porphyrins, planar bis-N-donor heterocyclic bases, metal chelator tethered intercalators and natural product small molecules.

14. The method of claim 13, wherein the DNA-binding metal chelating agent is complexed to a transition metal selected from the group consisting of iron, copper, manganese, nickel, zinc, ruthenium, rhodium and cobalt.

15. The method of claim 13, wherein the DNA-binding metal chelating agent is a porphyrin.

16. The method of claim 15, wherein the porphyrin is selected from the group consisting of meso-tetra (6-methyl-N-methyl-2-pyridyl) porphyrin, meso-α,β,γ-tritolyl-δ-(N-methyl-4-pyridiniumyl) porphyrin (1+), meso-α,β-ditolyl-γ,δ-(N-methyl-4-pyridiniumyl) porphyrin (cis-2+), meso-α,γ-ditolyl-β,δ-di (N-methyl-4-pyridiniumyl) porphyrin (trans-2+), meso-α-tolyl-β,γ,δ-tri (N-methyl-4-pyridiniumyl) porphyrin (3+) and tetra (N-methyl-4-pyridyl) porphyrin.

17. The method of claim 16, wherein the porphyrin is tetra (N-methyl-4-pyridyl) porphyrin.

18. The method of claim 17, wherein the tetra (N-methyl-4-pyridyl) porphyrin is complexed to manganese (III).

19. The method of claim 13, wherein the DNA-binding metal chelating agent is a planar bis-N-donor heterocyclic base selected from the group consisting of 1,10-phenanthroline, dipyridoquinoxaline and dipyridophenazine.

20. The method of claim 19, wherein the bis-N-donor heterocyclic base is 1,10-phenanthroline.

21. The method of claim 20, wherein the 1,10-phenanthroline is complexed to copper (II).

22. The method of claim 13, wherein the DNA-binding metal chelating agent is a metal chelator tethered intercalator selected from the group consisting of acridine porphyrins, acodazole porphyrins and methidiumpropyl EDTA.

23. The method of claim 22, wherein the metal chelator tethered intercalator is methidiumpropyl EDTA.

24. The method of claim 23, wherein the methidiumpropyl EDTA is complexed to iron (III).

25. The method of claim 13, wherein the DNA-binding metal chelating agent is a natural product small molecule.

26. The method of claim 25, wherein the natural product small molecule is selected from the group consisting of bleomycin, adriamycin, leinamycin, kanamycin, phleomycin and neamine.

27. The method of claim 11, wherein the backbone cleavage agent comprises an oxidant.

28. The method of claim 27, wherein the oxidant is selected from the group consisting of Oxone®, hydrogen peroxide, molecular oxygen, t-butyl hydroperoxide, peracetic acid, magnesium monoperoxyphthalate, iodosobenzoic acid and persulfate salts.

29. The method of claim 28, wherein the oxidant is Oxone®.

30. The method of claim 28, wherein the oxidant is molecular oxygen.

31. The method of claim 30, wherein molecular oxygen is utilized in the presence of a reductant.

32. The method of claim 31, wherein the reductant is selected from the group consisting of ascorbate salts, 3-mercaptopropionic acid, β-mercaptoethanol and dithiothreitol.

33. The method of claim 12, wherein the treatment with a backbone cleavage agent comprises the following sequential steps:
a. treatment with a DNA-binding metal chelating agent; and
b. treatment with an oxidant, provided that the oxidant is not molecular oxygen.

34. A kit that is suitable for use in the isolation of a biomolecule originating from natural, genetically engineered or biotechnological biological sources, which is substantially free from intact nucleic acids, comprising the following steps:
a. treatment of a medium comprising the biomolecule to be isolated with a solution comprising a small molecule nucleic acid cleavage agent; and
b. purification of the resultant medium comprising the biomolecule to remove damaged DNA and/or the small molecule nucleic acid cleavage agent;
wherein the kit comprises a small molecule nucleic acid cleavage agent, chromatographic matrices for purification of a biomolecule and aqueous buffer solutions.

35. The kit of claim 34, wherein the small molecule nucleic acid cleavage agent comprises a DNA-binding metal chelating agent and an oxidant.

36. The kit of claim 35, wherein the DNA-binding metal chelating agent is tetra (N-methyl-4-pyridyl) porphyrin.

37. The kit of claim 36, wherein the tetra (N-methyl-4-pyridyl) porphyrin is complexed to manganese (III).

38. The kit of claim 35, wherein the oxidant is Oxone®.
